(19) United States
(12) Patent Application Publication
(10) Pub. No.: US 2020/0032194 A1
(43) Pub. Date: Jan. 30, 2020

(54) METHOD OF MEASURING MICROBIAL COUNT

(71) Applicant: JNC CORPORATION, Tokyo (JP)

(72) Inventors: Hajime TERAMURA, Kanagawa (JP); Ryuzo KIMURA, Tokyo (JP); Kojiro SOTA, Kanagawa (JP)

(73) Assignee: JNC CORPORATION, Tokyo (JP)

(21) Appl. No.: 16/336,910

(22) PCT Filed: Sep. 11, 2017
(86) PCT No.: PCT/JP2017/032605

§ 371 Date: Mar. 27, 2019

(30) Foreign Application Priority Data
Sep. 28, 2016 (JP) 2016-189545

(51) Int. Cl. C12M 1/34; C12N 1/20; C12Q 1/04

(52) U.S. Cl. CPC C12M 41/36; C12Q 1/045; C12N 1/20

(57) ABSTRACT

A method of measuring simply and accurately a microbial count in a sample. Namely, a method of measuring a microbial count including a step of blending a composition for preparing a culture medium for a microbial count measurement including (a) a polymer able to form a non-flowable transparent gel without a melting step by heating and without cooling, and (b) a nutrient component, with a sample added thereto, a step of incubating microorganisms contained in the sample, and a step of measuring the colony count of the microorganisms.

### TECHNICAL FIELD

The present invention relates to a method of measuring simply and easily a microbial count in a sample.

### BACKGROUND ART

With respect to drinking water, soft drink, industrial water, pharmaceutical water, dialysis water, etc., it is required that the level of contamination with should be minimal, and it is known that ordinarily the count of microorganisms present therein is very low. Especially, there are standards on a viable cell count of 100 CFU/mL or less with respect to drinking water, and 10 CFU/mL or less with respect to dialysis water. In controlling to the standards, monitoring for assessing periodically an accurate microbial count is important (Non Patent Literature 1).

For detecting a microorganism in a food or the like, ordinarily a method by which 1 mL of a sample suspension or the like is subjected to a pour culture is adopted. However, with respect to a sample such as drinking water in which the count of microorganisms present therein is very low, a microbial count may not be measured accurately by the above method. For example, in a case where 10 CFU or less of microbes are present in 100 mL, if only 1 mL of sample is examined, a microorganism may not be detected so that contamination situation may not be assessed accurately.

For eliminating such a drawback, a membrane filter method has been heretofore used. Namely, the total amount of a liquid sample is filtrated through a membrane filter to capture microorganisms in the sample on the surface of membrane filter and then incubated on an agar medium plate (Non Patent Literature 2). Even a low count of microorganisms present in a large volume of sample may be measured accurately by the method.

### CITATION LIST

### Non Patent Literature

NPL 1: The 17th Edition of The Japanese Pharmacopeia, General Information, G8 Water
NPL 2: “Toseki Seijou-ka Guideline (Guideline for Cleaner Dialysis), Ver. 2.01, 11 Mar. 2014, by Japan Association for Clinical Engineers.

### SUMMARY OF INVENTION

### Technical Problem

However, for the membrane filter method, equipment for filtration, including a funnel and a suction pump, is necessary, and it is troublesome to sterilize each piece each time. Further, certain skills are necessary for operation, because if a bubble is formed between a membrane filter and an agar medium surface in placing a membrane filter after filtration on an agar medium, there occurs a problem that some captured microbes may not touch the culture medium, and not be in a position to be detected.

In view of such circumstances, an object of the present invention is to provide a method of measuring simply and accurately a microbial count in a liquid sample, especially a low count of microorganisms present in a large volume of liquid sample, without using a special instrument.

### Solution to Problem

The present inventors studied diligently for achieving the object to arrive at simplification of operations by utilizing a liquid sample itself as a solvent composing a culture medium, and visualization of microorganisms in a sample by developing colonies through incubation. Further, it has been found that a polyacrylic acid and/or a salt thereof is a suitable gelling agent for a component of such a culture medium from the viewpoint of simple operation and easy visual recognition in performing a microbial count measurement, thereby completing the present invention.

Namely, the present invention is as follows.

[1] A composition for preparing a culture medium for a microbial count measurement comprising (a) a polymer able to form a non-flowable transparent gel without a melting step by heating and without cooling, and (b) a nutrient component (hereinafter referred to as a “composition of the present invention”).

[2] The composition according to [1] above, wherein the polymer is able to retain water 10 times or more as much as the own weight.

[3] The composition according to [1] or [2] above, wherein water separation of the gel does not take place.

[4] The composition according to any one of [1] to [3] above, wherein the polymer has acrylic acid as a monomer unit.

[5] The composition according to [4] above, wherein the polymer is a polyacrylic acid and/or a salt thereof

[6] The composition according to any one of [1] to [5] above comprising further (c) a color reagent.

[7] A kit for measuring a microbial count comprising the composition according to any one of [1] to [6] above, and a culture container.

[8] A method of measuring a microbial count comprising a step of blending the composition according to any one of [1] to [6] above with a sample added thereto,

a step of incubating microorganisms contained in the sample, and
a step of measuring the colony count of the microorganisms (hereinafter referred to as a “measuring method of the present invention”).

[9] The method according to [8] above, wherein the microbial count in the sample is 0.1 CFU/mL or less.

[10] The method according to [8] or [9] above, wherein the sample weight is 10 to 10000 times as high as the weight of the polymer in the composition.

[11] A use of a composition comprising (a) a polymer able to form a non-flowable transparent gel without a melting step by heating and without cooling, and (b) a nutrient component, for measuring a microbial count.

[12] A use of (a) a polymer able to form a non-flowable transparent gel without a melting step by heating and without cooling, and (b) a nutrient component in the manufacture of a composition for preparing a culture medium for a microbial count measurement.

Although there is no particular restriction on a sample hereunder, it is ordinarily a liquid sample, and is specifically an aqueous liquid sample, such as drinking water, soft drink, industrial water, pharmaceutical water, dialysis water, and urine.

Further, a microorganism means hereunder ordinarily coliform group, staphylococci, Vibrio bacteria, enterococci, fungi, Bacillus subtilis, etc.

### Advantageous Effects of Invention

The microbial count in a sample may be measured simply and accurately according to the present invention. Especially, even a low count of microorganisms present in a large volume of sample may be successfully detected quantitatively.

### BRIEF DESCRIPTION OF DRAWINGS

FIG. 1 is a photograph of one embodiment in which a composition of the present invention is used in a bag type container (100 mL capacity).

FIG. 2 is a photograph of red colonies in 100 mL of a gel in Example 1.

### DESCRIPTION OF EMBODIMENTS

A composition of the present invention mandatorily contains (a) a polymer able to form a non-flowable transparent gel without a melting step by heating and without cooling, and (b) a nutrient component.

A composition of the present invention is for preparing a culture medium for a microbial count measurement. The preparation is carried out ordinarily by adding a liquid sample containing measure target microorganisms as it is, as a solvent for a gel composing a culture medium, and blending them together. In such a usage mode, a composition of the present invention and a culture medium prepared using the same are different from a conventional culture medium for a microorganism.

From another viewpoint, the present invention may be regarded as a use of a composition comprising (a) a polymer able to form a non-flowable transparent gel without a melting step by heating and without cooling, and (b) a nutrient component, for measuring a microbial count.

Also, the present invention may be regarded as a use of (a) a polymer able to form a non-flowable transparent gel without a melting step by heating and without cooling, and (b) a nutrient component in the manufacture of a composition for preparing a culture medium for a microbial count measurement comprising.

A (a) polymer able to form a non-flowable transparent gel without a melting step by heating and without cooling assumes a role of a gelling agent composing a culture medium for incubation and measurement of a target microorganism. The polymer forms the gel by being blended with a liquid sample.

As the polymer, one able to retain water preferably 10 times or more as much as the own weight, more preferably 20 times or more, and further preferably 30 times or more, is suitable. Through such retention of water, a gel suitable for preparation of a culture medium may be formed.

Since a formed gel is non-flowable, the abundance of microorganisms may be measured accurately. Further, it is preferable that water separation does not occur in the gel. If water separation occurs, although existence of a colony of a microorganism is qualitatively detectable, the abundance may be occasionally hardly measured accurately. In this regard, water separation means water retained in a gel is separated from the gel. Further, “water separation does not occur” means specifically, for example, the amount of water to be separated from a gel after standing at room temperature for 60 min is preferably 0.5% or less, and more preferably 0.1% or less of the initial water amount retained therein.

Since a gel to be formed is transparent, a colony of a microorganism may be visually detected accurately. In this regard, “transparent” means that a visible light transmittance found by a spectrophotometric measurement (optical path length: 1 cm), when a polymer is added into distilled water at a concentration at which a gel to be formed is not flowable, is preferably 70% or more (with respect to the visible light transmittance of distilled water as 100%), but not limited thereto.

Since the polymer is able to form a gel without a melting step by heating and without cooling, the operation becomes simple and growth of a target microorganism is not impeded. In this regard, “heating” means herein to elevate a temperature from room temperature, and specifically to elevate a temperature to a range where a microorganism is not viable, for example, beyond 60° C. Meanwhile, “cooling” means to cool down a polymer from the temperature at which it was dissolved in a liquid sample. Further, “room temperature” means hereunder ordinarily from 1 to 40° C., preferably from 1 to 30° C., and further preferably from 20 to 30° C.

Preferable examples of such a polymer include those having acrylic acid as a monomer unit, and insofar as it has acrylic acid as a monomer unit, it may be not only a homopolymer, but also it may be a copolymer, or even a crosslinked polymer. Specifically, it is preferably a polyacrylic acid and/or a salt thereof, or a derivative thereof, and especially sodium polyacrylate is appropriate.

Sodium polyacrylate is one of so-called super absorbent polymers, and is able to solidify a liquid at room temperature without heating or cooling, and furthermore able to form a uniform solid state by a simple mixing operation such as mild shaking. Therefore, a preparation operation for a culture medium is simple and easy.

A gel produced with sodium polyacrylate has a high transparency. Further, since sodium polyacrylate is available at a low cost, it is a gelling agent appropriate for the present invention.

Generally, for a microbial culture medium, etc., a gelling agent, such as agar, carrageenan, and locust bean gum, is used, for which heating is necessary in solidifying a liquid solvent uniformly. Therefore, the above is not appropriate for solidifying a liquid sample, which contains a microorganism, as it is. Further, it is not appropriate also because a gel solidified using the gelling agent has a low transparency.

Meanwhile, poly(vinyl alcohol) has drawbacks in that it is hardly miscible uniformly with a liquid solvent and it is apt to cause water separation. Further, with respect to xanthan gum, it is also difficult to mix the same with a liquid solvent, and it is apt to develop lumps and further to make a solidified gel nontransparent.

Since carboxymethylcellulose is unable to solidify a liquid sample and a flowable gel is formed, the same is not appropriate for quantitative detection of microorganisms.

Meanwhile, hyaluronic acid is able to form a transparent gel having a high water retention capacity and a low flowability, therefore it is applicable to the present invention. However, from the aspect of cost, polyacrylic acid and/or a salt thereof is more preferable.

In a case where sodium polyacrylate is used as (a) a polymer according to the present invention, a degree of polymerization of 10,000 or more is preferable from the viewpoint of solidification power, and a degree of polymerization of 22,000 or more is more preferable. It may be crosslinked, or not.

Although there is no particular restriction on the concentration of sodium polyacrylate during use according to the present invention, it is preferably, for example, 10 g to 0.01 g/100 mL, and more preferably 5 g to 0.5 g/100 mL.

In a case where another (a) polymer is used, the concentration during use may be in any range insofar as a solid gel is formed, and to the extent that the advantages of the present invention are not impaired.

A (b) nutrient component is used for developing a target microorganism.

Although there is no particular restriction on a nutrient component, preferable examples thereof include peptone, an animal meat extract, a yeast extract, and a fish meat extract.

As described in Non Patent Literature 1, it is recommended to use a standard agar medium for a test of drinking water, and an R2A agar medium for a test of pharmaceutical water, and dialysis water. Therefore, it is preferable to use a broth medium of the agar media, from which agar is eliminated, or an equivalent component as a component of a composition of the present invention.

A composition of the present invention preferably comprises further (c) a color reagent. This is for making a microbial colony, to be formed by incubation, colored, so that its detection and measurement can become easier.

Examples of a color reagent include an oxidation-reduction indicator, such as 2,3,5-triphenyltetrazolium chloride (TTC), and tetrazolium Violet. The same may be used favorably for a case where all kinds of microorganisms present in a sample should be measured. When TTC is used, its concentration during use is preferably 100 mg to 1 mg/L, and more preferably 50 to 10 mg/L.

As a color reagent, a compound, which is a substrate with respect to an enzyme owned solely by a specific kind of microorganism (hereinafter referred to as “enzyme substrate”) and is able to release a colorant compound by degradation, may be used. The same may be used favorably for measuring a specific microorganism.

In this regard, as a colorant compound, any kind that is colored under visible light, or emits fluorescence may be used. Examples of a functional group to be released as a colored compound under visible light include a 5-bromo-4-chloro-3-indoxyl group. A released 5-bromo-4-chloro-3-indole is converted through oxidation condensation to 5,5′-dibromo-4,4′-dichloro-indigo to develop a blue color. Examples of a functional group to be released as a fluorescent compound include 4-methylumbelliferyl group. A released 4-methylumbelliferone emits fluorescence under irradiation with ultraviolet light.

Examples of a favorably usable enzyme substrate include, in a case where a target microorganism is a coliform group, 5-bromo-4-chloro-3-indoxyl-beta-D-galactopyranoside (X-GAL), and 5-bromo-4-chloro-3-indoxyl-beta-D-glucuronic acid; in a case of yellow staphylococcus, 5-bromo-4-chloro-3-indoxyl phosphate (X-phos); in a case of enterococcus 5-bromo-4-chloro-3-indoxyl-beta-D-glucopyranoside (X-GLUC); and in a case of fungus, X-phos, 5-bromo-4-chloro-3-indoxyl acetate, and 5-bromo-4-chloro-3-indoxyl butyrate. Further, in a case where all kinds of microorganisms should be detected, all of the above may be used in a combination.

The concentration of the enzyme substrates during use is preferably, for example, from 0.01 to 1.0 g/L, and more preferably from 0.2 to 1.0 g/L.

A composition of the present invention may further comprise a selective agent, an antibacterial substance, inorganic salts, saccharides, a thickener, a pH adjuster, etc.

Examples of a selective agent include an antibiotic, such as polymyxin B and vancomycin, and a surfactant, such as sodium lauryl sulfate (SDS), Tween 80, and a bile salt including sodium cholate.

Examples of an antibacterial substance include polylysine, protamine sulfate, glycine, and sorbic acid.

Examples of inorganic salts include an inorganic acid metal salt, such as sodium chloride, and sodium thiosulfate, and an organic acid metal salt, such as sodium pyruvate, ferric ammonium citrate, and sodium citrate.

Examples of saccharides include glucose, lactose, sucrose, xylose, cellobiose, and maltose.

Examples of a thickener include starch and a derivative thereof, hyaluronic acid, an acrylic acid derivative, polyether, and collagen.

Examples of a pH adjuster include sodium carbonate, and sodium hydrogen carbonate. In this regard, a composition of the present invention is composed such that the pH during use becomes preferably from 6.0 to 8.0 from the viewpoint of growth of a target microorganism, and more preferably from 6.5 to 7.5.

A composition of the present invention may be offered in a combination with a culture container as a kit for measuring a microbial count.

The culture container is for receiving a liquid sample ordinarily as it is without a treatment, such as concentration and dilution, and mixing therein the liquid with a composition of the present invention allowing a polymer contained in the composition to gelate such that a culture medium is formed for incubating a microorganism.

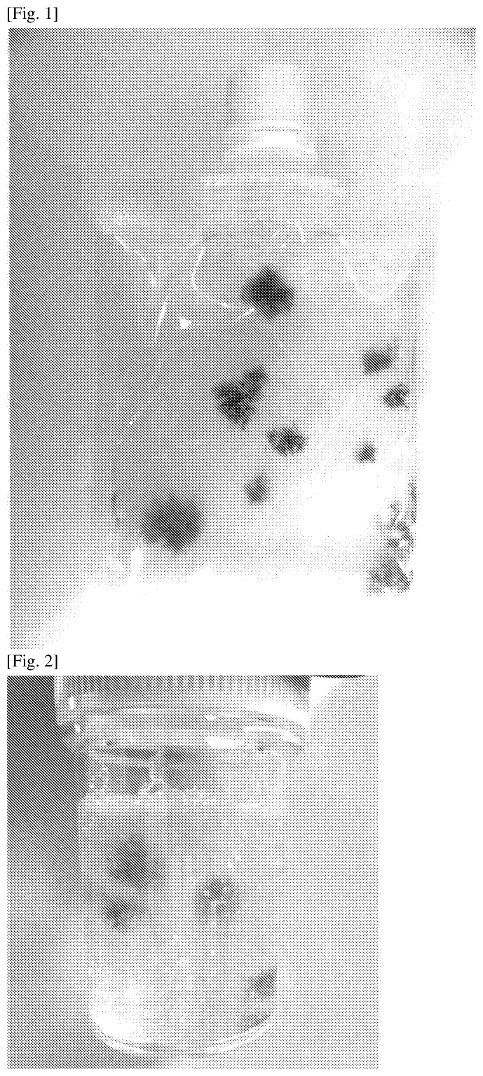

There is no particular restriction on the shape of a culture container, insofar as a necessary amount of a liquid sample may be accommodated adequately. For example, for blending a composition of the present invention and a liquid sample by shaking, a container having a shape such as cylindrical, and made of a material resistant to de-formation is preferable. Meanwhile, for example, in a case where a composition of the present invention and a liquid sample are blended by rubbing or squeezing together with a container, a container made of a flexible material, which is easily deformable, is preferable. Preferable examples thereof include a bag type container made of a polyvinyl-type polymer, or a polyethylene-type polymer, and a container with a closure, such as a cap, and a zipper, is more preferable (refer to FIG. 1). Further, a culture container is preferably transparent, because a microbial colony may be measured easily from the outside of the container. There is no particular restriction on the maximum receivable amount, and a preferable example is 100 to 1000 mL, which is suitable for application to a large volume of sample containing a small count of microorganisms.

In the case of a conventional kit, a sample is contacted with a culture medium prepared previously, and then incubation and measurement are performed. In contrast, a kit for measuring a microbial count of the present invention is suitable for a mode of use, in which the polymer is made to gelate using a liquid sample itself as a solvent in the culture container such that a microorganism in a sample is incubated in the gel and then measured.

A culture container may be also a small-sized plate (sheet), which is suitable for detection of a sample with a volume as small as approximately 1 mL.

Examples thereof include an ordinary dish, and a container type combining a dish-like concave sheet and a flat or convex sheet. By forming a flat shape, a microbial colony may be measured more easily. Further, by downsizing the same, a plurality of samples may be processed easily in a parallel way.

Since such a small-sized flat-shaped culture container may be applicable to a diluted sample, it is also suitable for a case where the microbial count in a sample is, for example, 300 CFU/mL or less.

A composition for preparing a culture medium for a microbial count measurement of the present invention described above may be used favorably for a measuring method of the present invention.

A measuring method of the present invention comprises a step of blending the composition of the present invention with a sample added thereto, a step of incubating microorganisms contained in the sample, and a step of measuring the colony count of the microorganisms.

A composition of the present invention and a liquid sample may be blended by an optional method, and for example they may be blended by shaking a container and its content altogether, squeezing the same, or stirring the content with a sterilized tool.

There is no particular restriction on incubation conditions for a microorganism, and they me be selected appropriately depending on the type of a target microorganism. For example, 35±2° C. and 24 to 48 hours are preferable.

In a culture medium after incubation grown colonies of a target microorganism appear, and may be detected visually or otherwise, and the count thereof may be measured accurately.

A measuring method of the present invention may be used favorably for a sample with a low abundance of a microorganism, namely a highly clean sample. For example, it is appropriate for a case where the microbial count in a sample is as low as 0.1 CFU/mL or less, which is not detectable by an ordinary inspection using 1 mL.

In general, with respect to a sample with a high abundance of a microorganism, a measurement may be performed after diluting the sample appropriately suited to a detection method, however with respect to a case with a low abundance, concentration may be troublesome or difficult. A measuring method of the present invention is useful even in such a case, because the microbial count can be detected simply and accurately.

Further, a measuring method of the present invention is very useful, because even a large amount of liquid sample can be subjected to measurement as it is without a pre-treatment. For example, it is appropriate in a case where a sample weight is large corresponding to the water retention capacity of (a) the polymer in a composition of the present invention, for example, in the case of sodium polyacrylate, is 10 to 10000 times as large as the polymer weight. Or more specifically, it is appropriate in a case where a sample is of a large volume, for example 100 mL or more.

There is no particular restriction on a sample to which a measuring method of the present invention is applicable, and examples thereof include a liquid sample, such as drinking water, soft drink, industrial water, pharmaceutical water, dialysis water, and urine. The same may be also applicable to a culture solution yielded by incubating the sample in advance with a tryptic soy broth, etc.

### EXAMPLES

Next, the present invention will be described in detail by way of Examples, provided that the present invention be not restricted by the Examples.

### Example 1

(1) Production of Composition for Preparing a Culture Medium for a Microbial Count Measurement

Source materials having a composition set forth in Table 1 were mixed in a 150 mL cylindrical transparent plastic container to produce a composition of the present invention.

TABLE 1

| Source material | Composition (g/100 mL) |
|---|---|
| Yeast extract | 0.05 |
| Peptone | 0.05 |
| Casamino acid | 0.05 |
| Glucose | 0.05 |
| Soluble starch | 0.05 |
| Sodium pyruvate | 0.03 |
| Potassium hydrogen phosphate | 0.03 |
| Magnesium sulfate heptahydrate | 0.005 |
| TTC | 0.0025 |
| Sodium polyacrylate | 1 |
| | pH 7.2 |

* As sodium polyacrylate, AQUALIC CA (Nippon Shokubai Co., Ltd.) was used.

(2) Strains for Test

Bacillus subtilis NBRC3134, and Escherichia coli NBRC102203 were used as strains for test. Each of them was precultured for 24 hours on a tryptic soy agar medium, and suspended in a sterile physiological saline solution using a sterile cotton swab to form a bacterial stock solution corresponding to McFarland nephelometric standard #1 (approximately 3.0×10⁸ CFU/mL). Using each bacterial stock solution, 10-fold serial dilution with a sterile physiological saline solution was repeated down to 10⁻⁸ to produce a bacterial diluted solution of several CFU/mL. One (1) mL of the bacterial diluted solution was added into 99 mL of sterile water to form a sample liquid with a microbial count as low as several CFU/100 mL. Each sample liquid in an amount of 100 mL each was added to a composition produced as above, and the mixture was shaken up and down for blending and solidified at room temperature. The solidified culture medium was left standing allowing incubation at 35° C. for 24 hours, and then examined whether development occurred, or not.

### Comparative Example 1 and Comparative Example 2

Likewise, respective compositions were prepared as Comparative Example 1 and Comparative Example 2 by blending 30 g of poly(vinyl alcohol) (weight-average molecular weight; 5000 to 200000, and degree of saponification: 85 to 90%), or 10 g of xanthan gum (molecular weight 2000000 or higher, Product Number of producer: 02960021, Producer: MP Biomedicals Inc., and Distributor: Wako Pure Chemical Industries, Ltd.) in place of sodium polyacrylate in Example 1. The strains were tested for evaluation identically with Example 1.

(Evaluation Result)

FIG. 2 shows colonies of Bacillus subtilis in Example 1.

In the case of Example 1, in which a composition of the present invention was used, a sample liquid with either of the bacterial strains solidified rapidly, and after incubation red colonies were recognized in a transparent gel as shown in FIG. 2, and the colony count could be easily measured.

On the other hand, in the case of Comparative Example 1 namely a composition using poly(vinyl alcohol) as a gelling agent, a sample liquid with either of the bacterial strains could solidify only partly and separation of a liquid component was recognized. Further, in the case of Comparative Example 2, in which xanthan gum was used as a gelling agent, xanthan gum itself clumped so that a liquid component could not solidify uniformly to form a large number of insolubles. Further, a solidified part was nontransparent.

### INDUSTRIAL APPLICABILITY

The microbial count in a sample may be measured simply and accurately according to the present invention. Especially, even a low count of microorganisms present in a large volume of sample may be successfully detected quantitatively. Therefore, even with respect to a highly clean sample such as drinking water, the count of a small number of microorganisms present therein can be measured thoroughly without fail. So, the present invention is useful.

1. A composition for preparing a culture medium for a microbial count measurement comprising (a) a polymer able to form a non-flowable transparent gel without a melting step by heating and without cooling, and (b) a nutrient component.

2. The composition according to claim 1, wherein the polymer is able to retain water 10 times or more as much as the own weight.

3. The composition according to claim 1,
wherein water separation of the gel does not take place.

4. The composition according to claim 1, wherein the polymer has acrylic acid as a monomer unit.

5. The composition according to claim 4, wherein the polymer is a polyacrylic acid and/or a salt thereof.

6. The composition according to claim 1 comprising further (c) a color reagent.

7. A kit for measuring a microbial count comprising the composition according to claim 1, and a culture container.

8. A method of measuring a microbial count comprising a step of blending a composition with a sample added thereto,
a step of incubating microorganisms contained in the sample, and
a step of measuring the colony count of the microorganisms,
wherein the composition comprises (a) a polymer able to form a non-flowable transparent gel without a melting step by heating and without cooling, and (b) a nutrient component.

9. The method according to claim 8, wherein the microbial count in the sample is 0.1 CFU/mL or less.

10. The method according to claim 8, wherein the sample weight is 10 to 10000 times as high as the weight of the polymer in the composition.

11. (canceled)

12. (canceled)

13. The method according to claim 8, wherein the polymer is able to retain water 10 times or more as much as the own weight.

14. The method according to claim 8, wherein water separation of the gel does not take place.

15. The method according to claim 8, wherein the polymer has acrylic acid as a monomer unit.

16. The method according to claim 15, wherein the polymer is a polyacrylic acid and/or a salt thereof.

17. The method according to claim 8, wherein the composition further comprises (c) a color reagent.
